(19) United States
(12) Patent Application Publication
(10) Pub. No.: US 2013/0190653 A1
(43) Pub. Date: Jul. 25, 2013

(54) DEVICE FOR BLOOD COLLECTION FROM THE PLACENTA AND THE UMBILICAL CORD

(71) Applicant: Angel Gabriel Alvarez Ramos, Gijon (ES)

(72) Inventor: Angel Gabriel Alvarez Ramos, Gijon (ES)

(21) Appl. No.: 13/688,739

(22) Filed: Nov. 29, 2012

Related U.S. Application Data
(63) Continuation-in-part of application No. 13/386,927, filed on Jan. 25, 2012.

(30) Foreign Application Priority Data
Oct. 18, 2012 (ES) 201231607

(51) Int. Cl. A61B 5/15

(52) U.S. Cl. CPC A61B 5/150099; A61B 5/150007; A61B 5/150053
USPC 600/575; 600/573

(57) ABSTRACT

A device is provided for blood extraction and collection from the placenta and/or the umbilical cord through a system that combines the collection of the blood falling under gravity, together with a suction system facilitating this fall. Thus, a larger amount of blood can be extracted than is obtained by conventional gravity systems without posing a health risk to the mother, allowing a controlled suction that prevents premature detachment of the placenta.

### BACKGROUND OF THE INVENTION

1. Field of the Invention

The invention relates to a device for extracting and collecting blood from the placenta and/or umbilical cord, more specifically to a device based on the collection of this blood by allowing it to fall under gravity in combination with a suction system enabling improvement of the collection process.

2. Description of the Related Art

In recent years, the discovery of the existence of a high content of stem cells in the umbilical cord and in the placenta has encouraged the development of various systems of extracting and collecting blood contained in these organs in order to store it for subsequent use, either for scientific aims or, if required, to be transplanted into patients suffering from certain congenital or acquired diseases such as immunodeficiencies, cancers or metabolic disorders of deposition.

For transplantation of blood from the umbilical cord and the placenta to be effective, providing the required therapeutic level in the pathologies to be treated, it is crucially important to obtain a sufficient volume of blood rich in stem cells, requiring at least in the order of 1.5×10⁷ precursor cells per kg of patient body weight for a successful transplant.

Currently, the vast majority of systems for blood extraction from the placenta and the umbilical cord are based on sterilised devices for puncturing the umbilical cord immediately after the expulsion of the neonate, during the minutes before the detachment of the placenta. The blood thus extracted falls under the effect of gravity into the blood collection bag, this bag being kept below the height of the woman who has just given birth, thereby encouraging the falling of the blood. Normally, the bag used includes anticoagulant substances and is kept in motion during the collection process, encouraging the preservation of the blood under optimal conditions.

Using the method described above, the average volume of blood collected in each delivery is estimated at approximately 120 ml. Although this volume is often sufficient to obtain a stem cell content that is later therapeutically effective, in a not insignificant number of patients this volume does not contain the stem cell content necessary as there are insufficient cell numbers.

Currently, the state of the art includes some systems for obtaining an increase in the blood extracted and collected from the placenta and the umbilical cord, mainly through pressure systems for aspirating this blood, thereby increasing the volume collected and reducing the collection time. Examples of these types of systems are the devices described in patents WO2005/041772, U.S. Pat. No. 5,097,842, U.S. Pat. No. 5,059,168 and U.S. Pat. No. 5,575,795. These devices include one or more needles for perforating the umbilical vein and/or placenta, means of collecting the blood such as reservoirs, bags or vacuum containers, and means of aspiration or drainage such as vacuum pumps or peristaltic pumps. In all the references cited above, increasing the amount of blood collected is achieved by replacing the usual method, based on a puncture and subsequent fall under gravity, by a method based exclusively on negative pressure exercised by means of suction or drainage.

Although the devices are capable of carrying out the purposes for which they were designed, producing an increase in the amount of blood collected, they share a common problem related to their basic design being exclusively based on a suction method. As a consequence of the continuous application of this suction, there is the possibility of inducing the appearance of complications such as the sudden appearance of retroplacental haematoma and, consequently, premature detachment of the placenta before detachment naturally takes place and the maternal homeostasis factors at the time of childbirth can minimise bleeding. This detachment, which may be precipitated by a constant uncontrolled suction action of blood, involves an obvious health risk to the mother.

It is therefore necessary to develop devices that enable increasing the volume of blood collected from the placenta and the umbilical cord, so that the content of extracted stem cells is sufficient to meet the therapeutic needs and, at the same time, are safe for the mother during the time of delivery, ensuring natural detachment of the placenta without any risk to health.

The present invention aims to satisfy this need by a device for blood collection that combines the usual system of falling under gravity together with the controlled assistance supplied by a suction system.

Another advantage of the present invention is that with a design that combines extraction by falling under gravity and extraction by suction, as much blood can be extracted from the umbilical cord as that contained in the placenta by only puncturing the umbilical cord. Not needing to puncture the placenta is especially important as such a puncture normally implies a risk of bacterial contamination that can be avoided with the device described here. Additionally, the possibility of collecting blood from the placenta through a perforation of the umbilical cord makes it possible to carry out blood collection immediately after the expulsion of the neonate without needing to wait for the expulsion of the placenta, which can occur in a time from 5 to 30 minutes after the birth of the neonate, implying an optimisation in the working time of the surgeon and the consequent cost savings.

### SUMMARY OF THE INVENTION

An object of the present invention is a device for blood collection from the placenta and the umbilical cord that combines collection of blood by falling under gravity and simultaneously a suction system that facilitates this falling.

This object is achieved by a first preferred embodiment of the invention consisting of a device for blood collection from the placenta and the umbilical cord comprising a means of blood extraction constituted by at least one needle or catheter and at least one tube or cannula through which the extracted blood circulates, this blood falling under the effect of gravity into a means of collection; and where the device also comprises a means of suction that is connected to the tube or cannula and is also connected to the means of blood collection through this tube or cannula. This provides an efficient way to increase the volume of blood collected from the placenta and/or the umbilical cord compared to the devices of the current state of the art, additionally offering a technical solution that enables controlling the pressure for suction of the blood, thus enabling an enormous reduction in the risks derived from excessive applied pressure, or from continuous pressure that is produced by the known devices for blood collection.

Preferably, the means of blood extraction include at least one needle or catheter and a tube or replacement cannula. In this way there is an exchangeable system directly coupled to the extraction device that can be used in case of need without having to replace any additional element that would imply a delay or complication in the collection process.

In another preferred embodiment of the present invention, the tube comprises at least one means of closure, for example through clamps or, alternatively, systems of by-pass valves operated either manually or automatically. This provides means of control and retention of the collected blood. In addition to the by-pass valve or clamp, the collection device can also comprise within the system of valves, for example, two- or more way shut-off valves and/or no-return or anti-return valves. This provides a control and retention system for collected blood that enables the optimum configuration of the elements of the device in its collection circuit so that the volume of blood obtained can be maximised, additionally ensuring that the collected blood remains inside the device, without returning to the umbilical cord or to the mother.

In a second preferred embodiment of the invention, the point of connection of the means of suction to the tube or cannula is located at a distance of 75% or less of the maximum length of this tube or cannula from the collection device or bag, being preferably located less than 50% of the maximum length of the cannula from this bag, and more preferably less than 30%. In this way, a suitable distance is achieved to ensure both a substantial increase in blood collected and the realisation of a gentle pressure not applied too close to the umbilical cord (which could cause undesirable excessive suction). However, depending on the specific features of the type of blood collection that is being carried out, the position of both the means of suction and the optional means of closure can vary, they being located, for example, close to the needle, close to the means of collection, in an intermediate region or in a combination of configurations of these positions.

In a preferred embodiment of the present invention, the means of collection contains anticoagulants. These preserve the collected blood under optimal conditions.

In a preferred embodiment of the present invention, the means of suction can be operated manually or automatically, preferably by a manually operated syringe. This makes the device adaptable to different functions, depending on the requirements of each collection process.

In a preferred embodiment of the invention, at least the collection device or bag is sterile and, in a more preferred embodiment, the whole of the collection device with all its components is sterile: needle(s), cannula(s), bag(s), valve(s), means of suction.

Other characteristics and advantages of the present invention will emerge from the description of the invention that follows, as well as from the embodiments illustrated in the accompanying figures.

### DESCRIPTION OF THE DRAWINGS

FIG. 1 shows schematically the first preferred embodiment of the device described in the present invention.

FIG. 2 shows schematically the second preferred embodiment of the device described in the present invention.

### DETAILED DESCRIPTION OF THE PREFERRED EMBODIMENTS

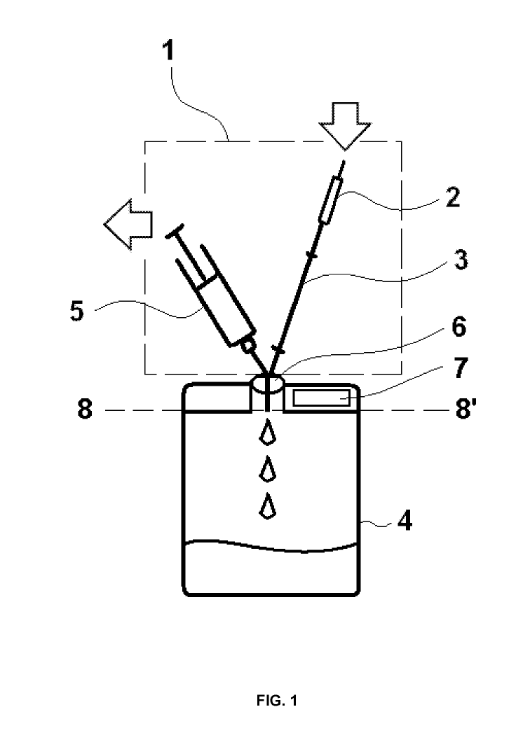

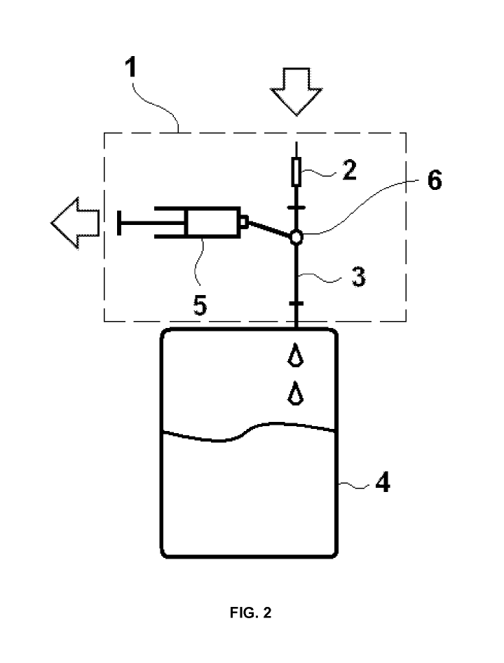

In accordance with the invention and according to the embodiments shown in FIGS. 1 and 2, the device for blood extraction and collection is constituted by at least one means of blood extraction (1) that preferably comprises at least one needle or catheter (2) for puncturing the umbilical vein and/or venous system of the placenta and at least one tube or cannula (3) through which the extracted blood flows. In different embodiments of the present invention it is possible to have more than one tube and needle as a replacement system in case of need. Each tube (3) is connected to at least one means of blood collection (4) that preferably comprises a collection bag, preferably incorporating anticoagulants for preserving the blood. Preferably, this bag is not subjected to prior vacuum.

The device of the present invention also comprises a means of suction (5) that is preferably constituted by a plunger system, operated manually or automatically. This means of suction is connected to the tube (3) and to the means of collection (4) through this tube (3).

Preferably, the tube (3) comprises at least one means of closure (6), for example by clamps or, alternatively, a by-pass valve system, operated manually or automatically. Additionally or alternatively to the by-pass valves, the collection device of the invention can also comprise closure valves of two- or more ways and/or non-return valves, for example, within the valve system. In this way, it is possible to have a device capable of exercising a negative pressure on the blood of the umbilical cord that carries the blood towards the means of suction (5) and that at the same time ensures that the collected blood is later kept inside the device, when the blood is transferred from this means of suction (5) towards the means of collection (4) by applying a positive pressure, reversing the movement of the plunger. In order to encourage non-return of the aspirated blood towards the umbilical cord, it is also possible to apply the means of suction (5) oriented substantially towards the means of collection (4) by, for example, an application route in the form of a “V”, arranged in the upper part of the means of collection (4) at the point of entry of this tube or cannula (3) into the means of collection (4) (see FIG. 1).

In an embodiment of the invention, the device or collection bag has in its upper part a zone (7) not to be filled with blood, arranged to house the entry cannula in the form of a “V”, coupled to the means of suction (5) and, eventually, with a two- or more way closure or by-pass valve. This zone (7) can be of the same material or a different material from the rest of the device or bag for blood collection. It is preferably made of a material that will allow writing on it the data about the collection, identity of the patient, etc. In this preferred embodiment of the invention, the mentioned zone (7) can allow the sticking of labels with the information referred to above, or can even have them already stuck or printed.

The point of connection of the means of suction (5) to the tube (3) is preferably located at a distance of substantially 75% or less of the maximum length of this tube (3) from the means of collection (4) or bag, being preferably located less than 50% of the maximum length of the tube (3) from said bag, and more preferably less than 30%, so that a substantial increase in the collected blood is obtained and a gentle pressure can be applied not too close to the umbilical cord. In different embodiments of the invention, depending on the specific characteristics of the type of blood collection being carried out, the position both of the means of suction (5) and of the optional means of closure (6) can vary, they being located, for example, close to the needle, close to the means of collection, in an intermediate region, or in a combination of configurations of these positions of these means (5, 6).

In the various embodiments of the invention, the blood collection device is preferably sterilized.

The blood collection procedure of the present invention thus comprises the following stages:
a) The umbilical vein and/or venous system of the placenta is punctured with a needle (2). Optionally, the umbilical vein can be punctured to extract the blood contained in the placenta without needing to puncture the latter.
b) The means of collection (4) is kept at a height below that of the mother and in constant motion encouraging respectively, the falling of blood under gravity and the preservation of the blood under optimal conditions.
c) The means of suction (5) is used to help the extraction of blood and its falling under gravity through the tube (3) towards said means of suction (5) in a controlled way, applying a sufficiently gentle negative pressure to prevent the risk of detachment of the placenta, the amount of this pressure being estimated at the discretion of the physician, depending on the characteristics of each patient and the circumstances of the delivery.
d) After carrying out the collection of a volume of blood towards the means of suction (5), for example until the volume of this means of suction (5) is filled, the pressure is reversed by the operation of the plunger, transferring the volume of blood towards the means of collection (4), where it is kept together with the rest of the volume of blood collected during the entire operation. Optionally, to carry out this step, it is possible to apply means of closure (6) to control the flow of blood from the means of suction (5) to the means of collection (4). These means of closure (6) can be any of those on the market and comprise any of the systems of valves described in the present invention: clamp, by-pass, many-way (at least two-way) valves, non-return valves, etc. The means of closure (6) of FIG. 1 can also consist of sealing off by simple folding/unfolding over of the means of collection or bag (4) in an embodiment of the invention in which the means of suction (5) is coupled in a “V” to the tube or cannula (3), in the region of its entry to the means of blood collection (4). The folding over of the means of collection (4) is preferably carried out along the line 8-8′ in FIG. 1.

By the process described above, it is possible therefore to draw residual blood in the placenta and the umbilical cord that is not accessible by a method based exclusively on falling under gravity. This enables a considerable increase in the volume of blood collected, as shown in the data of Table 1, where the results obtained in a sample of collections carried out with gravity collection bags are compared against the data obtained using the present device. Using the data in this table, it is possible to estimate the average increase in volume to be 51 ml when using the device for blood collection based on gravity plus suction of the present invention compared to the volume collected by the usual methods based on devices of gravity collection only. The best results were obtained with the second preferred embodiment of the invention of FIG. 2 (means of suction (5) coupled to the tube or cannula (3), at a short distance, but outside the blood collection device or bag (4)), that showed an average increase in the volume of blood collected over the first preferred embodiment of the invention of FIG. 1 (where the means of suction is coupled in a “V” to the tube or cannula (3) in the zone of entry to the means of collection (4)) of at least 10%.

TABLE 1

| | | Collection carried out |
|---|---|---|
| | Collection carried out | with the present |
| Group | under gravity alone | invention |
| Samples | 9749 | 10 |
| Mean (ml) | 120.1 | 158 |
| Standard deviation (ml) | 28.96 | 46.5 |
| Variance (ml) | 838.8 | 1946.6 |
| Median (ml) | 117 | 168 |
| 95% confidence | 119.5-121 | 125.5-192.1 |
| interval (ml) | | |

Furthermore, the existence of a means of suction in the device of the invention, that can be operated in a controlled way and designed to support the method of blood extraction and collection under gravity, is able to provide the guarantee that premature detachment of the placenta will not take place, so preventing the risks to the health of the mother that accompany the methods of collection by suction of the current state of the art.

All the embodiments described for the present invention should not be considered as limiting of other variations in the design or the materials employed in manufacture, provided that these variations do not alter the essence of the invention and the object thereof.

What is claim is:

1. A device for blood collection from the placenta and/or the umbilical cord comprising:
at least one means for blood extraction (1) constituted by at least one needle or catheter (2) and, at least, one tube or cannula (3) through which the extracted blood flows;
at least one means for blood collection (4) connected to the tube or cannula (3) and disposed so that the blood falls under gravity into the means of collection (4); and
means for suction (5) of blood connected to the tube or cannula (3) and also connected by the tube or cannula (3) to the means for blood collection (4).

2. The device for blood collection of claim 1, wherein the means for blood extraction (1) further comprises at least one replacement needle or catheter (2) and tube or cannula (3).

3. The device for blood collection of claim 1, wherein the tube or cannula (3) comprises at least one means for closing (6) the tube or cannula (3).

4. The device for blood collection of claim 3, wherein the means for closing (6) the tube or cannula (3) comprises at least one clamp.

5. The device for blood collection of claim 3, wherein the means for closing (6) the tube or cannula (3) comprises a system of by-pass valves (6) operated manually or automatically.

6. The device for blood collection of claim 3, wherein the means for closing (6) the tube or cannula (3) comprises one or more non-return valves.

7. The device for blood collection of claim 5, wherein the system of bypass valves (6) comprises at least one two- or more way valves.

8. The device for blood collection of claim 1, wherein the means for suction (5) is connected to the tube or cannula (3) at a distance of approximately 75% or less of a maximum length of the cannula from the means for collection (4).

9. The device for blood collection of claim 8, wherein the connection of the means for suction (5) to the tube or cannula (3) is at a distance of less than 50% of the maximum length of the tube or cannula (4) from the means for collection (4).

10. The device for blood collection of claim 9, wherein the connection of the means for suction (5) to the tube or cannula (3) is at a distance of less than 30% of the maximum length of the tube or cannula (4) from the means for collection (4).

11. The device for collecting blood of claim 1, wherein the means for suction (5) is connected to the tube or cannula (3) in a direction substantially orientated towards the means for collection (4), by an application route in the form of a “V” arranged in an upper part of the means for collection (4), at an entry of said tube or cannula (3) into the means for collection (4).

12. The device for blood collection of claim 11, wherein the means for collection (4) contains anticoagulants.

13. The device for blood collection of claim 11 wherein the means for suction (5) can be operated either manually or automatically.

14. The device for blood collection of claim 13, wherein the means for suction (5) is a manually operated syringe.

15. The device for blood collection of claim 11, further comprising a means for closure (6) of the tube or cannula (3) which comprise folding/unfolding over of the means for collection (4) in a region of the entry of the means for suction (5) and the tube or cannula (3) into the means for blood collection (4).

16. The device for blood collection of claim 11, further comprising a zone (7) arranged to house the entry cannula (3) in a “V”, the zone (7) being configured to additionally allow writing information or sticking information labels.

17. A method for blood collection from the placenta and/or the umbilical cord comprising:
a) puncturing the umbilical vein and/or venous system of the placenta with a needle (2);
b) connecting the needle (2) via a tube (3) to a means for collecting blood (4);
b) positioning the means for collecting blood (4) at a height below a position of puncturing the umbilical vein and/or venous system of the placenta to allow a gravitational flow of blood through the tube (3) and to the means for collecting blood (4);
c) operating a means for applying suction (5) that is connected to the tube (3) to help extract the blood that is falling gravitationally through the tube (3) and to draw at least part of the blood toward the means for suction (5); and
d) reversing the means for applying suction (5) to transfer the blood from a region of the means for applying suction (5) to the means for collecting blood (4).

18. The method of claim 17, further comprising selectively closing communication between the means for applying suction (5) and the means for collecting blood (4).

19. The method of claim 18, wherein the step of selectively closing communication between the means for applying suction (5) and the means for collecting blood (4) comprises folding the tube (3) or the means for collecting blood (4).

20. The method of claim 18, wherein the step of selectively closing communication between the means for applying suction (5) and the means for collecting blood (4) comprises operating a valve (6) connected to the tube (3) or to the means for collecting blood (4).
